(19) United States
(12) Patent Application Publication
(10) Pub. No.: US 2007/0016005 A1
(43) Pub. Date: Jan. 18, 2007

(54) Apparatus and method for recording the movement of organs of the body

(75) Inventors: Holger Timinger, Hamburg (DE); Sascha Kruger, Hamburg (DE); Hans-Aloy Wischmann, Henstedt-Ulzburg (DE); Jorn Borgert, Hamburg (DE); Jorg Sabczynski, Norderstedt (DE); Volker Rasche, Hamburg (DE)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., Eindhoven (NL)

(21) Appl. No.: 10/556,444

(22) PCT Filed: May 6, 2004
(86) PCT No.: PCT/IB04/50601

§ 371 Date: Jul. 12, 2006

(30) Foreign Application Priority Data
May 21, 2003 (EP) 03101455.8

(51) Int. Cl. A61B 5/05

(52) U.S. Cl. 600/424

(57) ABSTRACT

The invention relates to an apparatus and a method for recording the movement, caused in particular by breathing, of organs of the body such as the heart (9) for example. A part (3) of the diaphragm (10) is recorded by means of an X-ray device or an ultrasound device and the current position of the diaphragm is detected in the resulting image. Information about the associated position of other internal organs can be obtained from the position of the diaphragm with the aid of a model. This information can in turn be used, in a navigation system for a catheter, to set the spatial coordinates of the latter relative to the vascular system.

The invention relates to an apparatus and a method for recording the movement of internal organs such as, in particular, the heart. It furthermore relates to a navigation system for navigating a catheter in a vascular system.

When images are produced of an internal organ of a patient with the aid of an imaging apparatus, such as an X-ray device, at time intervals, it may be that the organ assumes different positions on the images. Causes of the organ displacement may be an overall movement of the patient and also in particular cyclic intrinsic movements caused by the breathing and heartbeat, the latter affecting in particular the organs of the chest and abdominal area. Displacement of the organ makes it considerably more difficult to compare X-ray images taken in a manner spaced apart over time.

Moreover, organ movements also disrupt navigation of a catheter in a patient's vascular system. The absolute spatial position of a catheter can be measured relatively well by means of appropriate locating devices. However, the position of the catheter relative to the vascular system or to the organs of the body is of great importance when navigating a catheter. Without knowing the movement of the organs, however, this position cannot be determined from the absolute position since it is affected by the superposition of individual movements.

In the literature, measurements have been described which can be used to examine the functional relationship between the movement of a person's diaphragm and the displacement of organs such as the heart (K. Nehrke, P. Boernert, D. Manke, J. C. Boeck: “Free-Breathing Cardiac MR Imaging: Study of Implications of Respiratory Motion—Initial Results”, Radiology, 220:810-815, 2001). The measurement of organ positions in this case takes place by means of special navigation rays of a nuclear magnetic resonance (NMR) device. However, NMR devices are very complex and expensive, so that their supplementary use in other clinical examinations is not conceivable.

Against this background, it is an object of the present invention to provide means for recording the movement of internal organs of the body, which means are relatively simple and cost-effective and are therefore suitable as a supplement to existing examination devices for example of a catheter laboratory.

This object is achieved by an apparatus having the features of claim 1, by a navigation system having the features of claim 9 and by a method having the features of claim 10. Advantageous refinements are given in the dependent claims.

The apparatus according to the invention is used to record the movement of at least one internal organ of the body. The movement may be caused by an overall movement of the patient examined, but particularly by cyclic intrinsic movements of the body such as heartbeat and breathing. The internal organ may be, for example, the heart. The apparatus comprises the following components:

a) An X-ray device and/or an ultrasound device for producing a one-dimensional or multidimensional (X-ray or ultrasound) image of at least one clearly defined body structure. A “clearly defined body structure” is in this case a body part, an organ, an organ border or the like and is shown clearly in the selected imaging mode in as sharply defined a manner as possible. In particular, the clearly defined body structure may be the diaphragm or a part thereof.

b) A data processing device which is coupled to the X-ray device (where present) and the ultrasound device (where present) and is designed to quantitively determine the position of the clearly defined body structure in an image produced by the X-ray device or ultrasound device and then to generate from this position a movement parameter which describes the movement of at least one internal organ of the body. In the simplest case, the movement parameter corresponds to the measured position of the clearly defined structure.

Such an apparatus thus makes it possible to obtain a movement parameter which can be used to correlate temporally offset images of body organs and/or to locate a catheter relative to a body organ. In this connection, it is particularly advantageous that an X-ray device or ultrasound device which forms part of the standard equipment of many examination laboratories is used to obtain the parameter.

If the apparatus comprises an X-ray device, it may in particular be designed to carry out the imaging of the clearly defined body structure with a minimum size of the irradiation field and/or with a minimum dose of radiation. This ensures that the radiation to which the patient is exposed during production of the image is kept to a minimum. The X-ray device may comprise automatically adjustable collimators for limiting the extent of the irradiation field to a minimum and placing it such that the clearly defined body structure is well covered.

If the apparatus comprises an ultrasound device, the latter is preferably designed to produce at least one sectional image that contains the clearly defined body structure. The ultrasound device is preferably designed such that it can produce one to four different sectional images of the clearly defined body structure. In this case, the individual sectional images may in particular be perpendicular to one another in order to show the body structure in various spatial dimensions in section.

If the apparatus comprises an ultrasound device, it may furthermore have means for fixing the ultrasound device to the body of a patient and a locating device for determining the spatial position (position and orientation) of the ultrasound device, said locating device being coupled to the data processing device. In this embodiment of the apparatus, the ultrasound device may be fixed to the body of a patient so that it goes along with the overall movement of said patient. The images of internal organs or of a clearly defined structure that are produced by the ultrasound device therefore represent only “internal” intrinsic movements of the organs of the body, which are caused for example by breathing and heartbeat. The overall movement of the patient can in this case be recorded separately by the locating device. According to one preferred refinement of the apparatus, the latter is designed to produce images of alternating clearly defined body structures. The X-ray device or the ultrasound device is in this case controlled such that from time to time (for example after a certain number of images of a first clearly defined body structure have been produced) the observation window is placed on another clearly defined body structure. Such a change in the imaging window is particularly advantageous when using an X-ray device, since it prevents a specific region of the body from being overly exposed to radiation. The alternating clearly defined body structures may in particular be different parts of the diaphragm.

Furthermore, the data processing device is preferably designed to calculate a quality measure for the movement parameter generated by it. The quality measure expresses how certainly and how accurately it has been possible to determine the movement parameter and may be displayed to the user for example as a number or graph. The quality measure may also be taken into account during automatic evaluation of the movement parameter, for example by movement parameters of high quality being assigned a greater weight than those of low quality.

In a preferred development of the apparatus, the data processing device is designed to calculate the position of internal organs of interest of the body with the aid of a model, the model receiving the determined movement parameter as an input variable. In the event of intrinsic movements of the body, such as the breathing for example, the relative position of the body organs can be described particularly well by a model, where individual parameters of the model can preferably be adapted individually to a patient and the changing state of the model is recorded by a movement parameter as variable. In this way, an observation taken at a specific point of the body (e.g. the diaphragm) can be used to deduce the relative position of organs of the body that are further away (e.g. the heart).

The invention furthermore relates to a navigation system for controlling a catheter in a vascular system, where the term “catheter” in this connection is to be understood in a general sense and encompasses any instrument which is to be moved through the vascular system of a body. The navigation system comprises the following components:

a) A locating device for determining the spatial position (position and preferably also orientation) of the catheter. The locating device may comprises for example a magnetic field sensor attached to the catheter, said magnetic field sensor using, for position determination purposes, a magnetic field impressed on the space by a field generator.

b) An apparatus of the type mentioned above for determining a movement parameter. That is to say that the apparatus comprises an X-ray device and/or an ultrasound device by means of which an image of a clearly defined body structure can be produced, wherein a data processing device determines the position of the clearly defined body structure in the image and from this generates a movement parameter that describes the movement of internal organs.

c) A data processing device which is coupled to the locating device and to the apparatus according to feature b) and is designed to determine the position of the catheter relative to the vascular system. This data processing device and that of the apparatus according to b) may in this case be implemented by the same hardware.

The navigation system achieves the object of measuring, as precisely as possible, the position of a catheter moved in the body of a patient relative to the vascular system or to an organ of interest. In this case, in terms of measurement technology, only the use of a locating device for determining the absolute spatial position of the catheter and also an X-ray device or ultrasound device is necessary. Such devices, like a data processing device for controlling the taking and processing of images, are present as standard in almost every catheter laboratory or can be easily obtained. The production of the above-described navigation system therefore essentially requires only the appropriate connection of the existing components and also a programming of the data processing device so that it carries out the desired steps.

The invention furthermore relates to a method of recording the movement of internal organs of the body, in particular of the heart. The method comprises the following steps:

a) Producing an image of at least one clearly defined body structure by means of X-ray radiation and/or ultrasound.

b) Determining the position of the abovementioned clearly defined body structure in the image and generating a movement parameter which describes the movement of the body organ of interest.

The method thus includes, in a general manner, the steps that can be carried out by the apparatus described above. For details regarding the refinement, advantages and developments of the method reference should therefore be made to the explanations given above.

The invention will be further described with reference to examples of embodiments shown in the drawings to which, however, the invention is not restricted. The same references denote the same components in all figures.

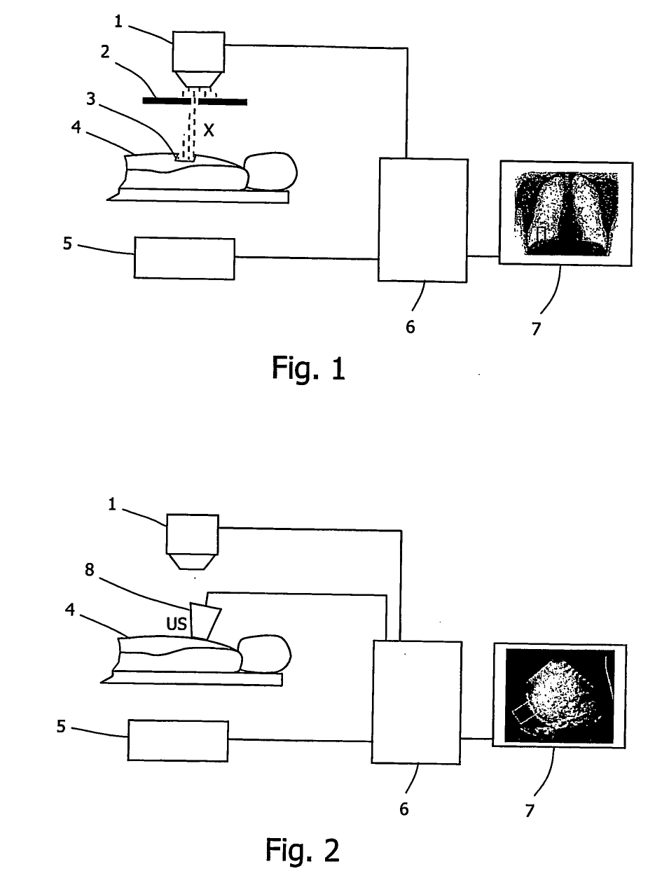

FIG. 1 shows an apparatus according to the invention for recording an organ movement by means of an X-ray device.

FIG. 2 shows an apparatus according to the invention for recording an organ movement by means of an ultrasound device.

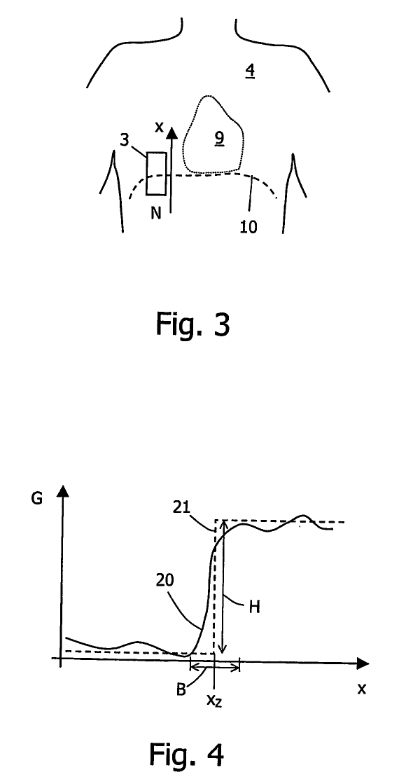

FIG. 3 shows a view of a patient's thorax with a diagram of an X-ray window recording the diaphragm.

FIG. 4 shows a one-dimensional X-ray image, obtained from the recording situation of FIG. 3, for locating the position of the diaphragm.

FIG. 1 schematically shows, in side view, a structure which can be used to record the movement of an internal organ of a patient 4. The patient 4 is located on a bed between an X-ray radiation source 1 and an associated X-ray detector 5. The X-ray radiation source 1 and the X-ray detector 5 are typically attached to a C-arm (not shown) and connected to a data processing device 6 (computer) for the purpose of controlling and reading the images. The data processing device 6 is coupled to a monitor 7 on which the image produced by the X-ray device can be displayed. The X-ray device furthermore has collimators 2 which can be adjusted by motors (not shown), the positioning of which collimators can be used to limit the X-rays X generated by the X-ray radiation source 1 to a desired irradiation window 3.

FIG. 3 shows in this respect a view of the thorax of the patient 4, with the positions of the diaphragm 10 and of the heart 9 being shown schematically. The irradiation window 3, which is right-angled in this example, covers part of the diaphragm 10 approximately in the center. It extends with a long side in the x direction which runs from the foot to the head of the patient 4, with the short side perpendicular thereto having a width of N pixels. Of course the irradiation window could also have any other suitable shape instead of a rectangle.

The described arrangement may be used as a breathing sensor, which makes it possible for the movement of internal organs of a patient 4, such as the liver or the heart 9 for example, caused by breathing to be recorded in real time. A determination of the current breathing phase and the intensity thereof is necessary for various medical applications. One important example of this is the navigation of a catheter during coronary interventions using static road maps. In this case, the absolute catheter position measured by a, for example magnetic, locating device must be compensated with respect to intrinsic movements of the body that are caused by the heartbeat and breathing. As experimental investigations have shown, there is a close anatomical correlation between the position of the diaphragm 10 and the position, movement and shape of adjoining organs such as the liver or heart 9 for example. This correlation may be recorded in a model which comprises, as input variable, the position of the diaphragm 10. In other words, knowing the diaphragm position, the movement of organs of the body caused by breathing can be compensated with the aid of a suitable model.

In the system shown in FIG. 1, the position of the diaphragm 10 is determined by taking an X-ray image in the small irradiation window 3, said window being precisely positioned by adjusting the collimators 2 such that it detects the edge of the diaphragm 10 at a specific sagittal position. The patient 4 is exposed to a low amount of radiation since the area of the irradiation region 3 is small. An additional reduction in dose can be achieved by reducing the radiation intensity. Although this reduces the X-ray contrast of the X-ray image produced, even a low contrast is sufficient for a simple detection of the position of the diaphragm as long as the difference between the signals from image zones within and outside the diaphragm is above the noise level. The small area of the irradiation region 3 moreover leads to there being less scattered radiation than in images of usual field sizes. This reduction in disruptive scattered radiation may be used to further reduce the dose while maintaining the same imaging accuracy. Finally, the exposure to radiation for the patient 4 can also be reduced by changing the position of the irradiation window 3 after each image or after a certain number of images, so that the same body volume is not always exposed to X-ray radiation.

Herein below, an optional method for determining the position of the diaphragm 10 will be described with reference to FIG. 4. The N image dots of an X-ray image of the irradiation window 3, which lie next to one another in the transverse direction, are binned in a first step of the method to form a mean gray value. A one-dimensional profile of the gray values G determined in this way then remains in the x direction, said profile being represented by the curve 20 in FIG. 4. In this connection, the binning leads to a reduction in the noise level with a reduction factor of N^{0.5}. A curve 21 with two different levels can be adapted to the gray value profile 20 using a curve fitting algorithm. The step position x_{z} of this curve 21 and the width B of the transition zone between the low level of gray values G and the high level of gray values G in the original curve 20 can then be used to quantitively describe the current position x_{z} of the diaphragm 10. Furthermore, the height H of the gray value stage together with the noise level can be used to derive a quality measure for the determined diaphragm position x_{z}.

The accuracy which can in principle be achieved with the system is only restricted by the spatial resolution of the X-ray device, which is usually sufficiently high. By contrast to conventional breathing sensors, such as for example a marker on the sternum, a chest strap or the like, the described method is simpler to carry out and less prone to error. Furthermore, with the method there is no attempt to determine a breathing phase (requiring additional information for determining the movement and deformation of an organ of interest), but rather the effect of the breathing is determined directly with respect to a movement of the diaphragm, which in turn is closely related to the movement of the organ of interest (heart, liver, etc.). In particular, therefore, there is also no need for any additional information about the type of breathing (chest breathing, abdominal breathing) since the position of the diaphragm directly reflects the displacement of the adjoining organs.

FIG. 2 shows an alternative system for determining the position of the diaphragm. The same references as in FIG. 1 denote the same components, so that reference may be made to what has been stated above in this respect. By contrast with FIG. 1, the system of FIG. 2 comprises an ultrasound device 8 which is coupled to the data processing device 6. The ultrasound device 8 produces ultrasound images of the diaphragm, one of which is shown schematically on the monitor 7. The quantitative determination of the diaphragm position from the ultrasound image may take place in a similar manner to that described above with reference to FIGS. 3 and 4 for the X-ray image. By determining the diaphragm position by means of ultrasound, the patient 4 is not exposed to any X-ray radiation at all.

The use of the ultrasound device 8 is moreover also suitable for combination with methods which monitor the overall position of the body of the patient 4. Such methods may analyze for example the ultrasound signals arising from reflections of suitable body zones of the patient. Alternatively, an ultrasound device may also be fixed to the body of the patient 4 (for example by means of a strap), the ultrasound device then being monitored with the aid of an additional movement sensor or a locating device.

Furthermore, the method can be expanded by using 4D ultrasound images (i.e. a temporal sequence of 3D ultrasound data) such that it allows a patient-specific movement model of the breathing to be rapidly derived. The imaging volume of the 3D data on which this is based may in particular be such that it contains both the organ to which movement compensation is to be applied and the organ/organ part driving the movement model. In a preprocessing step, the connection between the organ/organ part driving the movement model and the actual organ can then be analyzed, i.e. the patient-specific model can be derived. During the intervention, the measurement of the “driving” organ/organ part by means of conventional ultrasound (sequence of 2D sectional images) or X-ray imaging with collimators (sequence of 2D projection images) as described above may then be sufficient to carry out movement compensation.

The ultrasound device 8 may furthermore also produce sectional images of an organ of interest, such as in particular the heart, from which sectional images the movement state or the position and shape of the organ can be determined directly and/or input parameters for a model can be derived. Preferably, in this connection, use is made of one to four ultrasound probes which produce sectional images that are oriented relative to one another such that a sufficiently accurate position determination of the organ of interest is possible. In particular, three of the sectional planes may be perpendicular to one another. The information, which can be derived from the ultrasound images, about the movement state of the heart produced by heartbeat, breathing and/or patient movement may be used in conjunction with various imaging methods, such as 3D RCA (Rotational Coronary Angiography) and CT, in order to correlate, in a geometrically correct manner, the position of an interventional instrument (catheter, etc.), determined using a, for example magnetic, locating device, with the images.

Besides the abovementioned correlation of positions of an instrument (for example a catheter), measured by a locating device, with recorded data records, a further field of application is the targeted administration of medicaments during treatment of a coronary disease.

1. An apparatus for detecting the position of an invasive instrument in relation to internal organs of the body, comprising:
a) a source of a location signal indicating the location of the invasive instrument;
b) an X-ray device and/or an ultrasound device for producing an image of at least one clearly defined body structure; and
b) a data processing device which is coupled to the X-ray device or ultrasound device and responsive to the location signal and is designed to determine the position (x_{z}) of the clearly defined body structure in the image and to generate a movement parameter there from.

2. An apparatus as claimed in claim 1, characterized in that the clearly defined body structure is a part of the diaphragm.

3. An apparatus as claimed in claim 1, characterized in that it comprises an X-ray device and is designed to produce an image of the body structure with a minimum size of the irradiation field sand/or with a minimum dose of radiation.

4. An apparatus as claimed in claim 1, characterized in that it comprises an ultrasound device which is designed to produce at least one sectional image that contains the clearly defined body structure.

5. An apparatus as claimed in claim 1, characterized in that it comprises an ultrasound device which has means for fixing it to the body of a patient, and in that the location signal source comprises a locating device for determining the spatial position of the ultrasound device, said locating device being coupled to the data processing device.

6. An apparatus as claimed in claim 1, characterized in that it is designed to produce images of alternating clearly defined body structures.

7. An apparatus as claimed in claim 1, characterized in that the data processing device is designed to calculate a quality measure for the movement parameter.

8. An apparatus as claimed in claim 1, characterized in that the data processing device is designed to calculate the position of an internal organ of the body with the aid of a model that is dependent on the movement parameter.

9. A navigation system for navigating a catheter in a vascular system, comprising
a) a locating device for determining the spatial position of the catheter;
b) an apparatus as claimed in claim 1 for determining a movement parameter; and
c) a data processing device which is coupled to the locating device and to the apparatus and is designed to determine the position of the catheter relative to the vascular system.

10. A method of recording the movement of internal organs of the body, comprising the steps
a) producing an image of at least one clearly defined body structure by means of X-ray radiation and/or ultrasound;
b) identifying the location of an invasive device in the body;
c) determining the position (x_{z}) of the clearly defined body structure in the image in relation to the invasive device and generating a movement parameter.
